(19) United States
(12) Patent Application Publication
(10) Pub. No.: US 2026/0250732 A1
(43) Pub. Date: Aug. 27, 2026

(54) POLYSACCHARIDES RESULTING IN IMPROVED TEXTURE OF DAIRY PRODUCTS

(71) Applicant: CHR. HANSEN A/S, Hoersholm (DK)

(72) Inventors: Vera Kuzina POULSEN, Hoersholm (DK); Paula GASPAR, Hoersholm (DK); Kristian JENSEN, Hoersholm (DK); Rute NEVES, Hoersholm (DK); Ahmad ZEIDAN, Hoersholm (DK)

(73) Assignee: CHR. HANSEN A/S, Hoersholm (DK)

(21) Appl. No.: 18/875,989

(22) PCT Filed: Jul. 6, 2023
(86) PCT No.: PCT/EP2023/068663

§ 371 Date: Dec. 17, 2024

(30) Foreign Application Priority Data
Jul. 8, 2022 (EP) 22183809.7

(51) Int. Cl. C12P 19/04; A23C 9/123; C08B 37/00; C12N 1/205; C12R 1/46

(52) U.S. Cl. CPC C12P 19/04; A23C 9/1238; C08B 37/006; C12N 1/205; C12R 2001/46

(57) ABSTRACT

The present invention relates to novel exocellular polysaccharides resulting in improved texture of dairy products and LAB strains producing such polysaccharides. The present invention also relates to methods of using the polysaccharides and/or the strains for making products and to products comprising the polysaccharides and/or the strains. Finally, the invention relates to methods for manufacturing the strains of the invention.

### FIELD OF THE INVENTION

The present invention belongs to the biotechnology field and relates to novel exocellular polysaccharides resulting in improved texture of dairy products and LAB strains producing such polysaccharides. The present invention also relates to methods of using the polysaccharides and/or the strains for making food products and to food products comprising the polysaccharides and/or the strains. Finally, the invention relates to methods for manufacturing the strains of the invention.

### BACKGROUND OF THE INVENTION

The texture of fermented milk is an important quality parameter, affecting consumer acceptance. The texture of fermented milk is dependent on both the bacteria used for fermentation and process parameters. Lactic acid bacteria (LAB) such as Streptococcus thermophilus strains providing good texture are beneficial for products such as yoghurt. Exocellular polysaccharides produced by bacteria can positively influence texture. The ability of LAB such as S. thermophilus to produce polysaccharides can result in improved texture of fermented products.

Even though the potential to synthesize exocellular polysaccharides is encoded within a genome, it would not always lead to polysaccharide production. Also, polysaccharide production would not always lead to increased texture in products such as food products. The amount, the type and size of polysaccharides and their interaction with proteins e.g., milk proteins are determining factors for texture development in products such as milk products e.g. fermented milk products.

Some LAB strains contribute significantly to an improved texture associated with their ability to produce exocellular polysaccharides. Exocellular polysaccharides can be produced both as unattached material excreted to the environment, which is often referred to as exopolysaccharides (EPS), or as a capsule attached to the surface of the bacteria referred to as capsular polysaccharides (CPS). EPS consist of either a single type of sugar (homo-exopolysaccharides) or repeating units made of different sugars (hetero-exopolysaccharides). EPS-producing LAB are of interest, since EPS act as natural viscosifiers and texture enhancers of fermented foods. Furthermore, EPS from food-grade LAB with defined rheological properties have potential for development and exploitation as food additives. EPS are known to improve the rheological properties (i.e., texture) of LAB-fermented products by influencing viscosity, syneresis, firmness and sensory properties. The primary structural features (monosaccharide type and configuration, glycosidic linkage, non-sugar decorations, charge), the conformation and molecular weight, the amount of polysaccharide and the interactions of the polysaccharide with other system components are all factors that can contribute to and influence the displayed techno-functional properties (Zeidan et al., (2017), Polysaccharide production by lactic acid bacteria: from genes to industrial applications. FEMS Microbiology Reviews, 41 (1), S168-S200). The gene cluster encoding for the enzymes responsible for the production of exocellular polysaccharides is commonly referred to as the eps gene cluster.

LAB have been shown to produce EPS through two pathways, encoded by distinct genomic components: the sucrase-dependent pathway and the Wzy-dependent pathway. S. thermophilus do not have a sucrose dependent pathway, and synthesize exocellular polysaccharides only through the Wzy-dependent pathway, which is encoded in a gene cluster, here referred to as an eps cluster

There is an extremely large variety of possible polysaccharide structures (identified by their repeating unit) determined by their sugar building blocks, anomeric configuration, glycosidic linkage, non-sugar decorations of the monosaccharides and conformation. The selection of suitable polysaccharides and polysaccharide-producing cultures to be used in fermented milk products depends on numerous factors such as functionality of the polysaccharide produced, product type and production process. For example, in the context of the present invention, suitable polysaccharides need to achieve the desired texture and must also fulfil other criteria to be commercially useful. Since texture is an important parameter for products, in particular for food and feed products such as fermented milk products, there is a need for further texturing polysaccharides and polysaccharide-producing LAB strains such as polysaccharide-producing S. thermophilus strains.

### SUMMARY OF THE INVENTION

The inventors have found strains producing exocellular polysaccharides, in particular EPS, that result in fermented milks with increased texture. The inventors have also elucidated the structure of the repeating unit of such polysaccharides. The exocellular polysaccharide structures of the present invention have not yet been reported. The present invention thus relates to novel and inventive exocellular polysaccharides, in particular EPS, and to LAB strains capable of producing them. The present invention also relates to compositions and starter cultures comprising such exocellular polysaccharides and/or strains as well as to methods of using the exocellular polysaccharides and/or strains for making products, such as food or feed products, and to products such as food or feed products comprising the exocellular polysaccharides and/or strains of the invention.

Hence, in a first aspect, the present invention relates to a polysaccharide, preferably an exocellular polysaccharide, even more preferably an EPS, with a repeating unit comprising one of the following backbones:

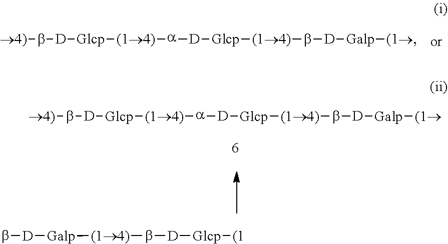

wherein the repeating unit comprising backbone (i) further comprises at least one side chain attached to any of the sugars in the backbone, wherein the side chain comprises or consists of structure (a):

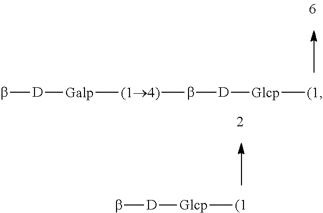

where (1->6) represents the linker to the monosaccharide in the backbone; and wherein the repeating unit comprising backbone (ii) further comprises at least one side chain attached to any of the sugars in the backbone, wherein the side comprises or consists of structure (b):

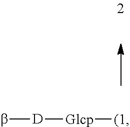

where (1->2) represents the linker to the monosaccharide in the backbone.

In a second aspect, the present invention provides a lactic acid bacterium (LAB) strain capable of producing the polysaccharides of the first aspect of the present invention, or a polysaccharide comprising a repeating unit which comprises or consists of one of the following structures:

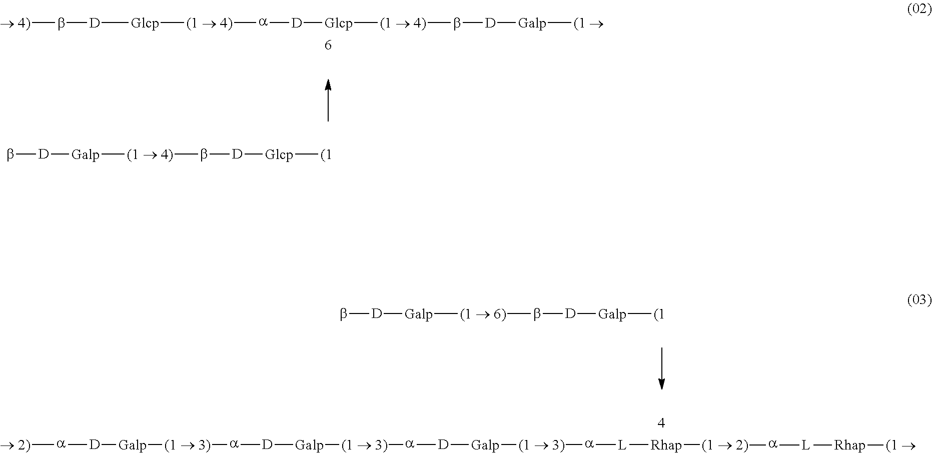

In a third aspect, the present invention relates to compositions and/or starter cultures comprising the polysaccharides of the first aspect and/or the LAB strain of the second aspect of the present invention.

In a fourth aspect, the invention relates to the use of the polysaccharides of the first aspect or a polysaccharide comprising a repeating unit which comprises or consists of one of the structures (2) and/or (3), the LAB strain of the second aspect and/or the compositions/starters of the third aspect of the present invention for improving texture of a product, e.g., for increasing viscosity of a product.

In a fifth aspect, the present invention also relates to methods of producing a product comprising a step in which the polysaccharides of the first aspect or a polysaccharide comprising a repeating unit which comprises or consists of one of the structures (2) and/or (3), the LAB strain of the second aspect and/or the compositions/starters of the third aspect of the present invention is used.

In a sixth aspect the present invention provides products comprising the polysaccharides of the first aspect or a polysaccharide comprising a repeating unit which comprises or consists of one of the structures (2) and/or (3), the LAB strain of the second aspect and/or the compositions/starters of the third aspect of the present invention.

In a seventh aspect the present invention provides methods for manufacturing LAB strains as defined in the present invention.

### DETAILED DESCRIPTION

### Definitions

All definitions of herein relevant terms are in accordance with what would be understood by the skilled person in relation to the herein relevant technical context.

In the context of the present invention in any of its embodiments, the expression “lactic acid bacteria” (“LAB”) designates food-grade bacteria producing lactic acid as the major metabolic end-product of carbohydrate fermentation. These bacteria are related by their common metabolic and physiological characteristics and are Gram positive, low-GC, acid tolerant, non-sporulating, rod-shaped bacilli or cocci. During the fermentation stage, the consumption of carbohydrate by these bacteria causes the formation of lactic acid, reducing the pH and leading to the formation of a protein coagulum. These bacteria are thus responsible for the acidification of milk and for the texture of the dairy product. The S. thermophilus strains of the present invention are classified as lactic acid bacteria.

By “texturing strain” in the present specification and claims is meant a strain which preferably increases the viscosity of fermented milks, more preferably which generates fermented milks having, under the conditions described below and as exemplified in Example 1 herein, a shear stress, measured at shear rate 300 s⁻¹, higher than 17 Pa, such as about 20 Pa, 25 Pa, 30 Pa, 35 Pa, 36 Pa, 36.5 Pa, 40 Pa, 41 Pa, 45 Pa, 50 Pa, 55 Pa, 60 Pa, 65 Pa, 66 Pa, 67 Pa, 70 Pa or more, in the presence of 0.2% yeast extract, and/or higher than 13.3 Pa, such as 13.5 Pa, 15 Pa, 20 Pa, 25 Pa, 30 Pa, 35 Pa, 40 Pa, 41.2 Pa, 41.4 Pa, 41.5 Pa, 42 Pa, 45 Pa, 50 Pa, 55 Pa, 60 Pa, 63 Pa, 64 Pa, 64.5 Pa, 65 Pa, 70 Pa or more, in the presence of 2% casein hydrolysate.

The texturing LAB strain (such as a S. thermophilus strain) of the invention may be an isolated strain, e.g., isolated from a naturally occurring source, or may be a non-naturally occurring strain, e.g., obtained recombinantly. Recombinant strains will differ from naturally occurring strains by at least the presence of the nucleic acid construct(s) used to transform or transfect the mother strain.

“Texture” is also an important quality factor for products, especially for fermented milk products. Consumer acceptance is often closely linked to texture properties. The texture of fermented milk is dependent on the exocellular polysaccharide structures, the bacteria used for fermentation and process parameters as well as milk composition. In the context of the present invention, the rheological properties (texture) of a fermented milk product, such as viscosity, can be measured as a function of “shear stress” of the fermented milk product by the following method (see also Example 1 for further details): When the pH of the fermented milk (e.g., mammalian- or plant-based milk) reaches pH~4.55 at the incubation temperature, e.g., 40° C., the fermented milk sample is cooled down by transferring the container to ice water (to, e.g., 4° C.). The fermented milk sample is manually stirred gently by means of a stick fitted with a perforated disc until homogeneity of the sample. In particular, the shear stress is measured as follows:
200 mL skim milk (38% protein, 53% lactose, <1.25 fat) is heated to 99° C. for 30 min, followed by cooling to inoculation temperature, and inoculated with 2 ml of an overnight culture of the lactic acid bacterium strain in the presence of 0.2% yeast extract or in the presence of 2% casein hydrolysate, and left at inoculation temperature until pH 4.55 and cooling down to 4° C., followed by gently stirring and measuring the shear stress at shear rate 300 s⁻¹, wherein the inoculation temperature is 40° C.

The rheological properties (i.e., texture) of a sample, such as the shear stress, are preferably assessed on a rheometer as described in Example 1, i.e., on an Anton Paar Physica Rheometer with ASC, Automatic Sample Changer, Anton Paar® GmbH, Austria by using a bob-cup. The rheometer is set to a constant temperature of 13° C. during the time of measurement. Settings are as follows:
Holding time (to rebuild to somewhat original structure);
5 minutes without any physical stress (oscillation or rotation) applied to the sample;
Oscillation step (to measure the elastic and viscous modulus, G′ and G″, respectively, therefore calculating the complex modulus G*)
   Constant strain=0.3%, frequency (f)=[0.5 . . . 8] Hz
   6 measuring points over 60 s (one every 10 s);
Rotation step (to measure shear stress at 300 1/s);
Two steps were designed:
Shear rate=[0.3-300] 1/s and 2) Shear rate=[275-0.3] 1/s.

Each step contains 21 measuring points over 210 s (on every 10 s). The shear stress at 300 1/s (300 s⁻¹) is chosen for further analysis, as this correlates to mouth thickness when swallowing a fermented milk product.

In the present context, the terms “strains derived from”, “derived strain” or “mutant” should be understood as a strain derived from a strain of the invention by means of, e.g., genetic engineering, radiation and/or chemical treatment, and/or selection, adaptation, screening, etc. It is preferred that the derived strain is a functionally equivalent mutant, e.g., a strain that has substantially the same, or improved, properties with respect to texturing properties, such as viscosity-enhancing properties, as the mother strain. Such a derived strain is a part of the present invention. Especially, the term “derived strain” or “mutant” refers to a strain obtained by subjecting a strain of the invention to any conventionally used mutagenization treatment including treatment with a chemical mutagen such as ethane methane sulphonate (EMS) or N-methyl-N′-nitro-N-nitroguanidine (NTG), UV light or to a spontaneously occurring mutant.

A mutant may have been subjected to several mutagenization treatments (a single treatment should be understood as one mutagenization step followed by a screening/selection step), but it is presently preferred that no more than 20, no more than 10, or no more than 5, treatments are carried out. In a presently preferred derived strain, less than 1%, or less than 0.1%, less than 0.01%, less than 0.001% or even less than 0.0001% of the nucleotides in the bacterial genome have been changed (such as by replacement, insertion, deletion or a combination thereof) compared to the mother strain.

The terms “fermented milk” and “dairy” are used interchangeably herein. In the context of the present invention in any of its embodiments, the expression “fermented milk product” means a food or feed product wherein the preparation of the food or feed product involves fermentation of a milk base with a lactic acid bacterium. “Fermented milk product” as used herein includes but is not limited to products such as thermophilic fermented milk products or mesophilic fermented milk products. The term “thermophilic fermentation” herein refers to fermentation at a temperature above about 35° C., such as between about 35° C. to about 45° C. The term “mesophilic fermentation” herein refers to fermentation at a temperature between about 22° C. and about 35° C.

In the context of the present application, the term “milk” is broadly used in its common meaning to refer to liquids produced by the mammary glands of animals (e.g., cows, sheep, goats, buffaloes, camel, etc.) or produced using plant bases. The term “milk base” or “milk substrate” may be any milk material that can be subjected to fermentation according to the present invention. In a preferred embodiment, the milk is cow's milk. In accordance with the present invention the milk may have been processed and the term “milk” includes whole milk, skim milk, fat-free milk, low fat milk, full fat milk, lactose-reduced milk, or concentrated milk. Fat-free milk is non-fat or skim milk product. Low-fat milk is typically defined as milk that contains from about 1% to about 2% fat. Full fat milk often contains 2% fat or more. Thus, useful milk bases include, but are not limited to, solutions/suspensions of any milk or milk like products comprising protein, such as whole or low-fat milk, skim milk, buttermilk, reconstituted milk powder, condensed milk, dried milk, whey, whey permeate, lactose, mother liquid from crystallization of lactose, whey protein concentrate, cream, or plant-based milks. The milk base may originate from any mammal, e.g., being substantially pure mammalian milk, or reconstituted milk powder. Plant sources of milk include, but are not limited to, milk extracted from soybean, pea, peanut, barley, rice, oat, quinoa, almond, cashew, coconut, hazelnut, hemp, sesame seed, sunflower seed and faba bean. Preferably, the plant-based milk is soy milk, which can be preferably supplemented with glucose, such as 0.5-5% glucose, 0.5-2% glucose, or about 2% glucose.

The term “product” as used herein refers to any product which would benefit from the addition of the exocellular polysaccharide as described herein. Preferably, the product is a food or feed product, more preferably a fermented food or feed product, even more preferably a dairy product, as described herein. The term “dairy product” as used herein refers to a food product produced from milk.

Prior to fermentation, the milk base may be homogenized and pasteurized according to methods known in the art.

“Homogenizing” as used in the context of the present invention in any of its embodiments, means intensive mixing to obtain a soluble suspension or emulsion. If homogenization is performed prior to fermentation, it may be performed to break up the milk fat into smaller sizes so that it no longer separates from the milk. This may be accomplished by forcing the milk at high pressure through small orifices.

“Pasteurizing” as used in the context of the present invention in any of its embodiments, means treatment of the milk base to reduce or eliminate the presence of live organisms, such as microorganisms. Preferably, pasteurization is attained by maintaining a specified temperature for a specified period of time. The specified temperature is usually attained by heating. The temperature and duration may be selected in order to kill or inactivate certain bacteria, such as harmful bacteria. A rapid cooling step may follow.

“Fermentation” in the context of the present invention in any of its embodiments means the conversion of carbohydrates into acids or alcohols or a mixture of both-through the action of microorganisms (LAB). Fermentation processes to be used in production of food products such as dairy products are well known and the person of skill in the art will know how to select suitable process conditions, such as temperature, oxygen, amount of microorganism(s) and process time. Fermentation conditions are selected so as to support the achievement of the present invention, e.g., to obtain a product, preferably a food or feed product which has improved texturing properties, such as improved viscosity properties, as compared to a product, preferably a food or feed product, produced with a method which does not involve the use of at least one of the EPS structures and/or strains producing such structures as described in the first aspect of the present invention or the use of strains or the composition as described in the present invention, in any of its embodiments.

As used herein, the term “about” (or “around”) means the indicated value±1% of its value, or the term “about” means the indicated value±2% of its value, or the term “about” means the indicated value±5% of its value, the term “about” means the indicated value±10% of its value, or the term “about” means the indicated value±20% of its value, or the term “about” means the indicated value±30% of its value; preferably the term “about” means exactly the indicated value (+0%).

Throughout the description and claims the word “comprise” and variations of the word (e.g., “comprising”, “having”, “including”, “containing”) typically is not limiting and thus does not exclude other features, which may be for example technical features, ingredients, substrates, components, or steps. However, whenever the word “comprise” is used herein, this also includes a special embodiment in which this word is understood as limiting; in this particular embodiment the word “comprise” has the meaning of the term “consist of”.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### The Polysaccharides of the Invention

In a first aspect, the present invention provides a polysaccharide, which is preferably an exocellular polysaccharide (EPS), which repeating unit comprises or, alternatively, consists of one of the following backbones:

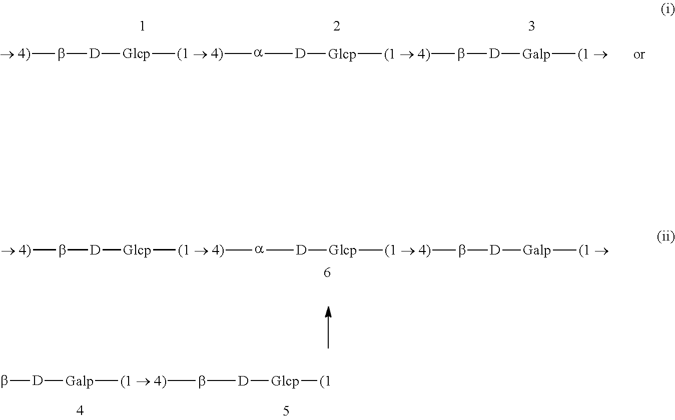

Preferably, the polysaccharide of the present invention additionally comprises one or more side chain(s) attached to one or more sugars of the backbone. Hence, the polysaccharide of the present invention is preferably a branched polysaccharide. The polysaccharide of the present invention is a heteropolysaccharide (HePS), since two or more types of sugars are present in the repeating unit.

In one embodiment, the polysaccharide of the present invention which repeating unit comprises or consists of the backbone (i) as defined above, comprises at least one side chain attached to any of the sugars in the backbone, wherein the side chain comprises or consists of the following structure:

(a)

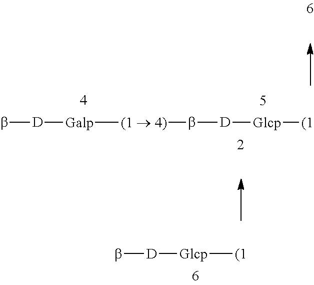

where (1->6) represents the linker to the monosaccharide in the backbone.

In one embodiment, the side chain as defined in (a) is attached to the α-D glucose present in backbone (i). Preferably, the side chain as defined in (a) is attached to the second α-D glucose of backbone (i), which corresponds to the monosaccharide labelled with number 2 in the above representation of backbone (i).

In one embodiment, the polysaccharide of the present invention which repeating unit comprises or consists of the backbone (ii) as defined above, comprises at least one side chain attached to any of the sugars in the backbone, wherein the side chain comprises the following structure:

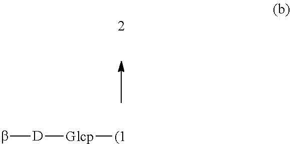

where (1->2) represents the linker to the monosaccharide in the backbone.

In one embodiment, the side chain as defined in (b) is attached to one of the D-glucoses present in backbone (ii). Preferably, the side chain as defined in (b) is attached to the α-D glucose of backbone (ii), which corresponds to the monosaccharide labelled with number 5 in the above representation of backbone (ii). In another embodiment, the side chain as defined in (b) is attached to the β-D-glucose and/or to the β-D-galactose of backbone (ii).

The side chain attached to backbone (i) or backbone (ii) of the polysaccharide of the present invention may comprise one or more sugars attached to the end of structures (a) or (b) as represented above, i.e., attached to the terminal β-D-Galp and/or β-D-Glcp of structure (a) or to the terminal β-D-Glcp of structure (b). In addition, the at least one side chain attached to backbone (i) of the polysaccharide of the present invention may comprise one or more sugars attached to the non-terminal monosaccharide, i.e., to the β-D-Glcp monosaccharide labelled with number 5 in the above representation of (a). Hence, the at least one side chain of the polysaccharide of the present invention may be a branched side chain. Such sugars attached to the side chain are preferably galactose and glucose, but may also be rhamnose, GalNAc, GlcNAc, ManNAc, fucose, ribose, non-ionic sugars or mixtures thereof. In one embodiment, the side chain attached to the backbone of the polysaccharide of the present invention comprises at least one extra sugar, preferably galactose or glucose, or mixtures thereof.

In one embodiment, the side chain of the polysaccharide of the present invention consists of structures (a) or (b) as represented above, i.e., the side chain attached to the backbone of the polysaccharide does not comprise any additional sugar residue.

In one embodiment, the polysaccharide of the present invention comprises a repeating unit which comprises or consists of the following structure:

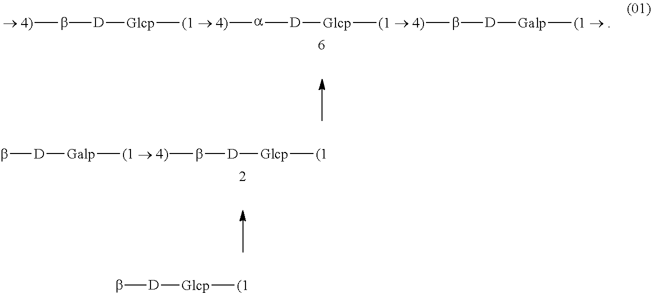

In some embodiments, the polysaccharide of the present invention comprises in its repeating unit one or more phosphate groups. In other embodiments, the polysaccharide of the present invention comprises in its repeating unit one or more substituent groups. These substitutional modifications include sulphonation, phosphorylation, acetylization, selenylation or combinations thereof.

LAB produce both polymers of high and low molecular weight. Polysaccharides synthesized via the Wzy-dependent pathway can range in molecular weight from 8 to over 10000 kDa. A simultaneous occurrence of polysaccharides of different sizes is frequent, i.e. LAB can produce simultaneously the same exocellular polysaccharide with different molecular weights, see Zeidan et al, 2017. In the present invention, the molecular weight (MW) of the polysaccharide of the present invention as described above is not particularly limited and may preferably be at least 1000 kDa, preferably in the range of from 1000 to more than 10000 kDa. For instance, the MW of the polysaccharide of the present invention is in the range of from 2000 to more than 10000 kDa, such as from 3000 to more than 10000 kDa, or from 4000 to more than 10000 kDa, or from 1000 to 8000 kDa, or from 2000 to 8000 kDa, or from 3000 to 8000 kDa, or from 4000 to 8000 kDa, or from 5000 to 8000 kDa, or from 1500 to 7000 kDa, or from 2000 to 7000 kDa, or from 2500 to 7000 kDa, or from 3000 to 7000 kDa, or from 4000 to 7000 kDa, or from 2500 to 6000 kDa, or from 3000 to 6000 kDa, or from 3000 to 5000 kDa.

Methods to determine the MW of exocellular polysaccharides such as EPS are well-known in the art. For instance, the polysaccharide can be isolated (e.g., as described in Example 2) and size-exclusion chromatography (SEC) can be used to determine the MW.

### The Lactic Acid Bacterium Strains of the Invention

In a second aspect, the present invention provides a lactic acid bacterium (LAB) strain capable of producing the polysaccharides of the first aspect of the present invention, or a polysaccharide comprising a repeating unit which comprises or consists of one of the following structures:

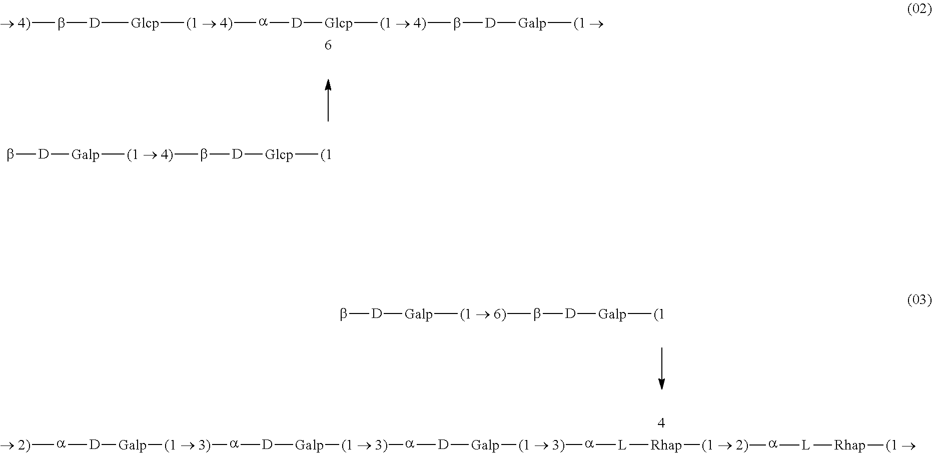

Preferably, the LAB strain of the present invention produces at least one of said polysaccharides. Preferably, the LAB strain of the present invention is a S. thermophilus strain.

More preferably, the LAB strain of the present invention is a strain:
(i) which generates fermented milks having a shear stress of more than 17 Pa, such as about 20 Pa, 25 Pa, 30 Pa, 35 Pa, 36 Pa, 36.5 Pa, 40 Pa, 41 Pa, 45 Pa, 50 Pa, 55 Pa, 60 Pa, 65 Pa, 66 Pa, 67 Pa, 70 Pa or more, measured at shear rate 300 s⁻¹ in the presence of 0.2% yeast extract, measured under following conditions:
   200 ml skim milk (38% protein, 53% lactose, <1.25 fat) is heated to 99° C. for 30 min, followed by cooling to inoculation temperature, and inoculated with 2 mL of an overnight culture of the lactic acid bacterium strain in the presence of 0.2% yeast extract, and left at inoculation temperature until pH 4.55 and cooling down to 4° C., followed by gently stirring and measuring the shear stress at shear rate 300 s⁻¹, wherein the inoculation temperature is 40° C.; and/or
(ii) which generates fermented milks having a shear stress of more than 13.3 Pa, such as 13.5 Pa, 15 Pa, 20 Pa, 25 Pa, 30 Pa, 35 Pa, 40 Pa, 41.2 Pa, 41.4 Pa, 41.5 Pa, 42 Pa, 45 Pa, 50 Pa, 55 Pa, 60 Pa, 63 Pa, 64 Pa, 64.5 Pa, 65 Pa, 70 Pa or more, measured at shear rate 300 s⁻¹ in the presence of 2% casein hydrolysate, measured under following conditions:
   200 mL skim milk (38% protein, 53% lactose, <1.25 fat) is heated to 99° C. for 30 min, followed by cooling to inoculation temperature, and inoculated with 2 mL of an overnight culture of the lactic acid bacterium strain in the presence of 2% casein hydrolysate, and left at inoculation temperature until pH 4.55 and cooling down to 4° C., followed by gently stirring and measuring the shear stress at shear rate 300 s⁻¹, wherein the inoculation temperature is 40° C.; and/or
(iii) which produces more than 1 μg/mL/OD₆₀₀ of EPS under the following conditions:
   Strains are grown in a Chemically Defined Medium supplemented with 2% lactose (w/v) under anaerobic conditions until stationary phase of growth (5-6 h) at 40° C. and constant pH 6.5; cell culture is then centrifuged (13,700×g, 30 min, 4° C.) and the supernatant collected and stored at −20° C. for isolation of EPS, as described in Example 2.

Preferably, the LAB strain of the present invention is a strain:
(i) which generates fermented milks having a shear stress of more than 35 Pa, such as about 36 Pa, 36.2 Pa, 36.5 Pa, 40 Pa, 41 Pa, 45 Pa, 50 Pa, 55 Pa, 60 Pa, 65 Pa, 66 Pa, 67 Pa, 70 Pa or more, measured at shear rate 300 s⁻¹ in the presence of 0.2% yeast extract, measured under following conditions:
   200 mL skim milk (38% protein, 53% lactose, <1.25 fat) is heated to 99° C. for 30 min, followed by cooling to inoculation temperature, and inoculated with 2 mL of an overnight culture of the lactic acid bacterium strain in the presence of 0.2% yeast extract, and left at inoculation temperature until pH 4.55 and cooling down to 4° C., followed by gently stirring and measuring the shear stress at shear rate 300 s⁻¹, wherein the inoculation temperature is 40° C.; and/or
(ii) which generates fermented milks having a shear stress of more than 40 Pa, such as 41.2 Pa, 41.4 Pa, 41.5 Pa, 42 Pa, 45 Pa, 50 Pa, 55 Pa, 60 Pa, 63 Pa, 64 Pa, 64.5 Pa, 65 Pa, 70 Pa or more, measured at shear rate 300 s⁻¹ in the presence of 2% casein hydrolysate, measured under following conditions:
   200 ml skim milk (38% protein, 53% lactose, <1.25 fat) is heated to 99° C. for 30 min, followed by cooling to inoculation temperature, and inoculated with 2 mL of an overnight culture of the lactic acid bacterium strain in the presence of 2% casein hydrolysate, and left at inoculation temperature until pH 4.55 and cooling down to 4° C., followed by gently stirring and measuring the shear stress at shear rate 300 s⁻¹, wherein the inoculation temperature is 40° C.

More preferably, the LAB strain of the present invention is a strain:
(i) which generates fermented milks having a shear stress of more than 40 Pa, such as about 40.9 Pa, 41 Pa, 45 Pa, 50 Pa, 55 Pa, 60 Pa, 65 Pa, 66 Pa, 67 Pa, 70 Pa or more, measured at shear rate 300 s⁻¹ in the presence of 0.2% yeast extract, measured under following conditions:
   200 ml skim milk (38% protein, 53% lactose, <1.25 fat) is heated to 99° C. for 30 min, followed by cooling to inoculation temperature, and inoculated with 2 mL of an overnight culture of the lactic acid bacterium strain in the presence of 0.2% yeast extract, and left at inoculation temperature until pH 4.55 and cooling down to 4° C., followed by gently stirring and measuring the shear stress at shear rate 300 s⁻¹, wherein the inoculation temperature is 40° C.; and/or
(ii) which generates fermented milks having a shear stress of more than 41 Pa, such as 41.2 Pa, 41.4 Pa, 41.5 Pa, 42 Pa, 45 Pa, 50 Pa, 55 Pa, 60 Pa, 63 Pa, 64 Pa, 64.5 Pa, 65 Pa, 70 Pa or more, measured at shear rate 300 s⁻¹ in the presence of 2% casein hydrolysate, measured under following conditions:
   200 ml skim milk (38% protein, 53% lactose, <1.25 fat) is heated to 99° C. for 30 min, followed by cooling to inoculation temperature, and inoculated with 2 mL of an overnight culture of the lactic acid bacterium strain in the presence of 2% casein hydrolysate, and left at inoculation temperature until pH 4.55 and cooling down to 4° C., followed by gently stirring and measuring the shear stress at shear rate 300 s⁻¹, wherein the inoculation temperature is 40° C.

Even more preferably, the LAB strain of the present invention is a strain:
(i) which generates fermented milks having a shear stress of more than 65 Pa, such as about 66 Pa, 66.9 Pa, 67 Pa, 70 Pa or more, measured at shear rate 300 s⁻¹ in the presence of 0.2% yeast extract, measured under following conditions:
   200 mL skim milk (38% protein, 53% lactose, <1.25 fat) is heated to 99° C. for 30 min, followed by cooling to inoculation temperature, and inoculated with 2 mL of an overnight culture of the lactic acid bacterium strain in the presence of 0.2% yeast extract, and left at inoculation temperature until pH 4.55 and cooling down to 4° C., followed by gently stirring and measuring the shear stress at shear rate 300 s⁻¹, wherein the inoculation temperature is 40° C.; and/or
(ii) which generates fermented milks having a shear stress of more than 64 Pa, such as 64.5 Pa, 64.9 Pa, 65 Pa, 70 Pa or more, measured at shear rate 300 s⁻¹ in the presence of 2% casein hydrolysate, measured under following conditions:
   200 ml skim milk (38% protein, 53% lactose, <1.25 fat) is heated to 99° C. for 30 min, followed by cooling to inoculation temperature, and inoculated with 2 mL of an overnight culture of the lactic acid bacterium strain in the presence of 0 2% casein hydrolysate, and left at inoculation temperature until pH 4.55 and cooling down to 4° C., followed by gently stirring and measuring the shear stress at shear rate 300 s⁻¹, wherein the inoculation temperature is 40° C.

See Examples 1 and 2 for further details.

Even more preferably, the LAB strain of the present invention is one of the following strains:
(i) Strain DSM 34289;
(ii) Strain DSM 34290;
(iii) Strain DSM 34291; or
   mutants or variants thereof with retained or further improved texturing properties, such as with retained or further improved shear stress properties.

As shown in the examples, strain DSM 34289 is capable of producing a polysaccharide comprising a repeating unit as follows:

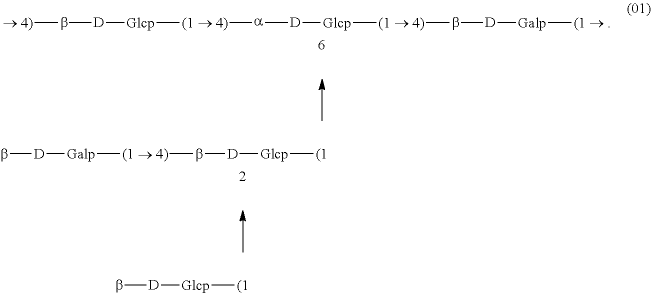

As shown in the examples, strain DSM 34290 is capable of producing a polysaccharide comprising a repeating unit as follows:

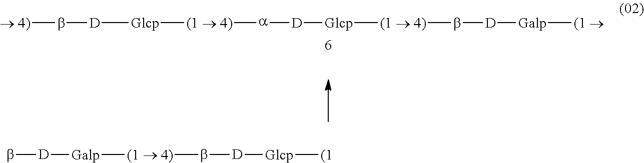

As shown in the examples, strain DSM 34291 is capable of producing a polysaccharide comprising a repeating unit as follows:

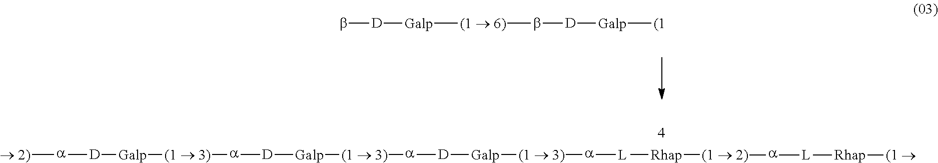

See Table 4.

The exocellular polysaccharides, preferably EPS, produced by the strains of the invention are involved in the excellent texturing capacity desirable in the production of fermented products, e.g., yoghurt, as described in detail below. As shown in Example 1, strains producing such exocellular polysaccharides (e.g., DSM 34289, DSM 34290, and/or DSM 34291) yield fermented milks with excellent texture.

Without being limited to theory, there is no substantial reason to believe that it would not be plausible that another LAB strain (i.e., different from the specific strains of the invention) that is able to produce at least one of the novel herein discussed exocellular polysaccharides, would not also have improved texturing properties when used for fermenting milk bases as described herein.

The Compositions and/or Starter Cultures of the Invention

In a third aspect, the present invention provides compositions and starter cultures comprising at least one polysaccharide and/or at least one LAB strain as defined in the first and second aspects of the invention, respectively.

Preferably, the composition or starter culture of the present invention in any of its embodiments comprises at least 1×10⁶ CFU (colony-forming units)/ml total LAB strains. It may be preferred that the composition comprises at least 1×10⁸ CFU/ml of the at least one, preferably one, lactic acid bacterium strain according to the invention.

In one embodiment, the composition or starter culture of the invention may also comprise at least one LAB strain according to the present invention and yeast extract, preferably yeast extract in an amount of about 0.2%. Yeast extract can be obtained from any source available to the skilled person, such as from Procelys (e.g., Yeast Extract NuCel® 545 MG, batch number 0005115910, batch AD 18 A05030). For example, the composition or starter culture of the present invention may comprise one or more of strains DSM 34289, DSM 34290, and/or DSM 34291 and yeast extract, preferably in an amount of 0.2%, as described above.

In one embodiment, the composition or starter culture of the invention may also comprise at least one LAB strain according to the present invention and casein hydrolysate, preferably casein hydrolysate in an amount of about 2%. Casein hydrolysate can be obtained from any source available to the skilled person, such as from Sigma-Aldrich (22090-500 G). For example, the composition of the present invention may comprise one or more of strains DSM 34289, DSM 34290, and/or DSM 34291 and casein hydrolysate, preferably in an amount of 2%, as described above.

As described above in the context of the second aspect of the present invention, the LAB strains of the present invention are able to generate fermented milks having improved texture, such as having high shear stress. Accordingly, the composition and/or starter culture of the present invention is able to generate at least the same shear stress as the one described for the LAB of the present invention.

LAB, including bacteria of the species S. thermophilus, are normally supplied to the dairy industry either as frozen (F-DVS) or freeze-dried (FD-DVS) cultures for bulk starter propagation or as so-called “Direct Vat Set” (DVS) cultures, intended for direct inoculation into a fermentation vessel or vat for the production of a dairy product, such as a fermented milk product. Such lactic acid bacterial cultures are in general referred to as “starter cultures” or “starters”. Accordingly, the composition of the present invention may be frozen or freeze-dried. In addition, the composition or starter culture of the present invention may be provided in liquid form. Thus, in one embodiment, the composition or starter culture is in frozen, dried, freeze-dried or liquid form.

The compositions or starter cultures of the present invention may also additionally comprise cryoprotectants, lyoprotectants, antioxidants, nutrients, fillers, flavorants or mixtures thereof. The composition preferably comprises one or more of cryoprotectants, lyoprotectants, antioxidants and/or nutrients, more preferably cryoprotectants, lyoprotectants and/or antioxidants and most preferably cryoprotectants or lyoprotectants, or both. Use of protectants such as cryoprotectants and lyoprotectants are known to a skilled person in the art. Suitable cryoprotectants or lyoprotectants include mono-, di-, tri- and polysaccharides (such as glucose, mannose, xylose, lactose, sucrose, trehalose, raffinose, maltodextrin, starch and gum arabic (acacia) and the like), polyols (such as erythritol, glycerol, inositol, mannitol, sorbitol, threitol, xylitol and the like), amino acids (such as proline, glutamic acid), complex substances (such as skim milk, peptones, gelatin, yeast extract) and inorganic compounds (such as sodium tripolyphosphate).

In one embodiment, the compositions or starter cultures according to the present invention may comprise one or more cryoprotective agent(s) selected from the group consisting of inosine-5′-monophosphate (IMP), adenosine-5′-monophosphate (AMP), guanosine-5′-monophosphate (GMP), uranosine-5′-monophosphate (UMP), cytidine-5′-monophosphate (CMP), adenine, guanine, uracil, cytosine, adenosine, guanosine, uridine, cytidine, hypoxanthine, xanthine, hypoxanthine, orotidine, thymidine, inosine and a derivative of any such compounds. Suitable antioxidants include ascorbic acid, citric acid and salts thereof, gallates, cysteine, sorbitol, mannitol, maltose. Suitable nutrients include sugars, amino acids, fatty acids, minerals, trace elements, vitamins (such as vitamin B-family, vitamin C). The composition may optionally comprise further substances including fillers (such as lactose, maltodextrin) and/or flavorants.

In one embodiment of the invention the cryoprotective agent is an agent or mixture of agents, which in addition to its cryoprotectivity has a booster effect.

The expression “booster effect” is used to describe the situation wherein the cryoprotective agent confers an increased metabolic activity (booster effect) on to the thawed or reconstituted culture when it is inoculated into the medium to be fermented or converted. Viability and metabolic activity are not synonymous concepts. Commercial frozen or freeze-dried cultures may retain their viability, although they may have lost a significant portion of their metabolic activity, e.g., cultures may lose their acid-producing (acidification) activity when kept stored even for shorter periods of time. Thus, viability and booster effect have to be evaluated by different assays. Whereas viability is assessed by viability assays such as the determination of colony forming units, booster effect is assessed by quantifying the relevant metabolic activity of the thawed or reconstituted culture relative to the viability of the culture. The term “metabolic activity” refers to the oxygen removal activity of the cultures, its acid-producing activity, i.e. the production of, e. g., lactic acid, acetic acid, formic acid and/or propionic acid, or its metabolite producing activity such as the production of aroma compounds such as acetaldehyde, α-acetolactate, acetoin, diacetyl and 2,3-butylene glycol (butanediol).

In one embodiment the compositions or starter cultures of the invention contains or comprises from 0.2% to 20% of the cryoprotective agent or mixture of agents measured as % w/w of the material. It is, however, preferable to add the cryoprotective agent or mixture of agents at an amount which is in the range from 0.2% to 15%, from 0.2% to 10%, from 0.5% to 7%, and from 1% to 6% by weight, including within the range from 2% to 5% of the cryoprotective agent or mixture of agents measured as % w/w of the frozen material by weight. In a preferred embodiment the culture comprises approximately 3% of the cryoprotective agent or mixture of agents measured as % w/w of the material by weight. The amount of approximately 3% of the cryoprotective agent corresponds to concentrations in the 100 mM range. It should be recognized that for each aspect of embodiment of the invention the ranges may be increments of the described ranges.

In a further aspect, the compositions or starter cultures of the present invention contains or comprises an ammonium salt (e.g. an ammonium salt of organic acid (such as ammonium formate and ammonium citrate) or an ammonium salt of an inorganic acid) as a booster (e.g. growth booster or acidification booster) for bacterial cells, such as cells belonging to the species S. thermophilus, e.g. (substantial) urease negative bacterial cells. The term “ammonium salt”, “ammonium formate”, etc., should be understood as a source of the salt or a combination of the ions. The term “source” of e.g. “ammonium formate” or “ammonium salt” refers to a compound or mix of compounds that when added to a culture of cells, provides ammonium formate or an ammonium salt. In some embodiments, the source of ammonium releases ammonium into a growth medium, while in other embodiments, the ammonium source is metabolized to produce ammonium. In some preferred embodiments, the ammonium source is exogenous. In some particularly preferred embodiments, ammonium is not provided by the dairy substrate. It should of course be understood that ammonia may be added instead of ammonium salt. Thus, the term ammonium salt comprises ammonia (NH₃), NH₄OH, NH₄⁺, and the like.

In one embodiment the composition of the invention may comprise thickener and/or stabilizer, such as pectin (e.g. HM pectin, LM pectin), gelatin, CMC, Soya Bean Fiber/Soya Bean Polymer, starch, modified starch, carrageenan, alginate, and guar gum.

Use of the Polysaccharides, Lab Strains and/or the Compositions/Starters for Improving the Texture of a Product

In a fourth aspect, the invention relates to the use of the polysaccharides of the first aspect or a polysaccharide comprising a repeating unit which comprises or consists of one of the structures (02) and/or (03), the LAB strain of the second aspect and/or the compositions/starters of the third aspect of the present invention for improving texture of a product, e.g., for increasing viscosity of a product. Preferably the product is a fermented product or a milk product, more preferably the product is a fermented milk product.

The molecular weight (MW) of the polysaccharide comprising a repeating unit comprising or, alternatively, consisting of the structure represented above as (02 and/or 03) is not particularly limited and may preferably be at least 1000 kDa, preferably in the range of from 1000 to more than 10000 kDa. For instance, the MW of the polysaccharide comprising a repeating unit comprising or, alternatively, consisting of the structure represented above as ((02 and/or 03) is in the range of from 2000 to more than 10000 kDa, such as from 3000 to more than 10000 kDa, or from 4000 to more than 10000 kDa, or from 1000 to 8000 kDa, or from 2000 to 8000 kDa, or from 3000 to 8000 kDa, or from 4000 to 8000 kDa, or from 5000 to 8000 kDa, or from 1500 to 7000 kDa, or from 2000 to 7000 kDa, or from 2500 to 7000 kDa, or from 3000 to 7000 kDa, or from 4000 to 7000 kDa, or from 2500 to 6000 kDa, or from 3000 to 6000 kDa, or from 3000 to 5000 kDa.

Hence, in the fourth aspect, the present invention provides a method for improving the texture (e.g., for increasing the viscosity) of a product, which is preferably a fermented product, preferably a milk product, more preferably of a fermented milk product, wherein the method comprises the use of the polysaccharide, the LAB strain, the composition and/or the starter culture of the present invention, as described in the first-third aspects of the present invention, or a polysaccharide comprising a repeating unit comprising or, alternatively, consisting of the structure represented as (02) and/or (03) above.

As described above, the LAB strains of the present invention as described in the first aspect and the compositions of the present invention, as described in the second aspect, are able to generate fermented milks having a shear stress greater than 17 Pa, such as about 20 Pa, 25 Pa, 30 Pa, 35 Pa, 36 Pa, 36.5 Pa, 40 Pa, 41 Pa, 45 Pa, 50 Pa, 55 Pa, 60 Pa, 65 Pa, 66 Pa, 67 Pa, 70 Pa or more, measured at shear rate 300 s⁻¹ in the presence of 0.2% yeast extract and/or greater than 13.3 Pa, such as 13.5 Pa, 15 Pa, 20 Pa, 25 Pa, 30 Pa, 35 Pa, 40 Pa, 41.2 Pa, 41.4 Pa, 41.5 Pa, 42 Pa, 45 Pa, 50 Pa, 55 Pa, 60 Pa, 63 Pa, 64 Pa, 64.5 Pa, 65 Pa, 70 Pa or more, measured at shear rate 300 s⁻¹ in the presence of 2% casein hydrolysate, measured as explained in Example 1.

In a specific embodiment of the third aspect, the present invention provides the use of S. thermophilus strain DSM 34289, S. thermophilus strain DSM 34290, and/or S. thermophilus strain DSM 34291, or mutants or variants thereof with retained or further improved shear stress properties, for improving the texture (e.g., for increasing viscosity) of a product, which is preferably a fermented product, preferably a milk product, more preferably of a fermented milk product.

### Product and Method for Producing a Product

The present invention further relates in a fifth aspect to methods of producing a product, such as a food or feed product, comprising a step in which at least one exocellular polysaccharide of the present invention, a polysaccharide comprising a repeating unit as defined in formula (02 and/or 03) above, the LAB strain and/or the composition or starter culture of the present invention are used. The production of the product, such as the food or feed product, is carried out by methods known to the person skilled in the art. In a sixth aspect, the invention relates to products, such as food or feed products, comprising the polysaccharides of the first aspect, a polysaccharide comprising a repeating unit as defined in formula (02 and/or 03) above, the LAB strain of the second aspect and/or the compositions/starters of the third aspect of the present invention.

The present invention further relates to a method for producing a product, such as a food or feed product, comprising at least one step in which at least one of the exocellular polysaccharide of the present invention and/or the polysaccharide comprising a repeating unit as defined in formula (02 and/or 03) above is used. The fifth aspect of the present invention also relates to a method for producing a product, such as a food or feed product, comprising at least one step in which at least one of the LAB strains of the present invention is used.

In another embodiment, the present invention relates to a method of producing a product, such as a food or feed product, comprising at least one step in which the lactic acid bacterium strain S. thermophilus DSM 34289 or a mutant or variant therefrom is used.

In another embodiment, the present invention relates to a method of producing a product, such as a food or feed product, comprising at least one step in which the lactic acid bacterium strain S. thermophilus DSM 34290 or a mutant or variant therefrom is used.

In another embodiment, the present invention relates to a method of producing a product, such as a food or feed product, comprising at least one step in which the lactic acid bacterium strain S. thermophilus DSM 34291 or a mutant or variant therefrom is used.

For instance, the method of the present invention comprises fermenting a milk substrate as described herein with a composition comprising at least 1×10⁶ CFU, preferably at least 1×10⁸ CFU/ml of at least one of the LAB strains of the present invention.

For instance, the method of the present invention comprises fermenting a milk substrate with a composition comprising at least 1×10⁶ CFU, preferably at least 1×10⁸ CFU/ml of strain S. thermophilus DSM 34289 or a mutant or variant therefrom.

For instance, the method of the present invention comprises fermenting a milk substrate with a composition comprising at least 1×10⁶ CFU, preferably at least 1×10⁸ CFU/ml of strain S. thermophilus DSM 34290 or a mutant or variant therefrom.

For instance, the method of the present invention comprises fermenting a milk substrate with a composition comprising at least 1×10⁶ CFU, preferably at least 1×10⁸ CFU/ml of strain S. thermophilus DSM 34291 or a mutant or variant therefrom.

In another preferred embodiment, the method comprises fermenting a milk substrate as described herein (also referred to as “milk base” in the context of the present invention) with the composition and/or starter as described in the present invention.

In another preferred embodiment, the method further includes the use of further lactic acid bacteria, such as other S. thermophilus strains or Lactobacillus bulgaricus strains and/or the use of casein hydrolysate and/or yeast extract, preferably at the concentrations indicated herein (about 2% casein hydrolysate or about 0.2% yeast extract).

The invention further provides products, such as food and feed products, comprising the polysaccharides of the first aspect, the polysaccharide comprising a repeating unit as defined in formula (02 and/or 03) above, the LAB strain of the second aspect and/or the compositions/starters of the third aspect of the present invention. Preferably, the products have been produced with the method of the fifth aspect of the present invention.

The product is preferably a fermented product. In another embodiment, the product is preferably a food or feed product, such as a milk product. Preferably, the product is a fermented food or feed product, such as fermented milk product. In a preferred embodiment, the product comprises and/or is made of plant-based and/or mammalian milk.

Hence, the food product may preferably be a dairy product, a meat product, a vegetable product, a fruit product or a cereal product. In a preferred embodiment, the food product is a dairy product, as defined above, such as yogurt or “tykmælk”. In another preferred embodiment, the food product is a plant-based (PB) food product, such as plant-based meat or dairy alternatives such as fermented soy milk or plant-based yoghurt (also referred to as “yogurt-like (YL) products” (PBYL)).

In a particular embodiment of the invention, the fermented milk product is selected from the group consisting of yoghurt, buttermilk, sour milk, cultured milk, Smetana, sour cream, thick cream, cultured cream, ymer, fermented whey, Kefir, Yakult and fresh cheese, such as Quark, tvarog and cream cheese. In a preferred embodiment, the fermented milk product is yoghurt. In a further preferred embodiment, the fermented milk product contains a further food product selected from the group consisting of fruit beverage, cereal products, fermented cereal products, chemically acidified cereal products, soymilk products, fermented soymilk products and any mixture thereof.

The fermented milk product typically contains protein in a level of between 1.0% to 12.0%, or between 2.0% to 10.0% by weight. In a particular embodiment, sour cream contains protein in a level of between 1.0% to 5.0%, or between 2.0% to 4.0% by weight. In a particular embodiment, Quark contains protein in a level of between 4.0% to 12.0%, or between 5.0% to 10.0% by weight.

Preferably, the product is a food product, more preferably is a dairy product and the method in any of its embodiments comprises fermenting a plant-based milk substrate (also referred to as “plant-based milk base” in the context of the present invention), such as soy milk, preferably soy milk supplemented with glucose, e.g., with 0.5-5% glucose, preferably 0.5-2% glucose, more preferably about 2%, with the at least one LAB strain and/or with the composition or starter culture according to the invention.

The food product according to the present invention may advantageously further comprise a “thickener” and/or a “stabilizer”, such as pectin (e.g. HM pectin, LM pectin), gelatin, CMC, Soya Bean Fiber/Soya Bean Polymer, starch, modified starch, carrageenan, alginate, and guar gum.

In another embodiment, the product is produced substantially free, or completely free of any addition of thickener and/or stabilizer. By substantially free should be understood that the product comprises from 0% to 20% (w/w) (e.g. from 0% to 10%, from 0% to 5% or from 0% to 2% or from 0% to 1%) thickener and/or stabilizer.

Preferably, the product has an improved texture (e.g., improved viscosity, measured as shear stress at 300 s⁻¹, as described in the present description and, e.g., in Example 1) as compared to a product produced with a comparable method which does not involve the use of at least one exocellular polysaccharide, the LAB strain and/or the composition or starter culture of the present invention.

### Methods for Manufacturing the Lab Strains

In a seventh aspect, the present invention provides methods for manufacturing a LAB strain, preferably the LAB strain of the present invention.

For instance, the present invention relates to a method for manufacturing a LAB strain wherein the method comprises the following steps:
(a) Providing a LAB strain, preferably a S. thermophilus strain, capable of producing exopolysaccharide (EPS) as defined in the first aspect of the present invention; and
(b) Screening for a LAB strain which is able to:
   (i) generate fermented milks having a shear stress of more than 17 Pa, such as about 20 Pa, 25 Pa, 30 Pa, 35 Pa, 36 Pa, 36.5 Pa, 40 Pa, 41 Pa, 45 Pa, 50 Pa, 55 Pa, 60 Pa, 65 Pa, 66 Pa, 67 Pa, 70 Pa or more, measured at shear rate 300 s⁻¹ in the presence of 0.2% yeast extract, measured under following conditions:
      200 ml skim milk (38% protein, 53% lactose, <1.25 fat) is heated to 99° C. for 30 min, followed by cooling to inoculation temperature, and inoculated with 2 mL of an overnight culture of the lactic acid bacterium strain in the presence of 0.2% yeast extract, and left at inoculation temperature until pH 4.55 and cooling down to 4° C., followed by gently stirring and measuring the shear stress at shear rate 300 s⁻¹, wherein the inoculation temperature is 40° C.; and/or
   (ii) generate fermented milks having a shear stress of more than 13.3 Pa, such as 13.5 Pa, 15 Pa, 20 Pa, 25 Pa, 30 Pa, 35 Pa, 40 Pa, 41.2 Pa, 41.4 Pa, 41.5 Pa, 42 Pa, 45 Pa, 50 Pa, 55 Pa, 60 Pa, 63 Pa, 64 Pa, 64.5 Pa, 65 Pa, 70 Pa or more, measured at shear rate 300 s⁻¹ in the presence of 2% casein hydrolysate, measured under following conditions:
      200 ml skim milk (38% protein, 53% lactose, <1.25 fat) is heated to 99° C. for 30 min, followed by cooling to inoculation temperature, and inoculated with 2 ml of an overnight culture of the lactic acid bacterium strain in the presence of 2% casein hydrolysate, and left at inoculation temperature until pH 4.55 and cooling down to 4° C., followed by gently stirring and measuring the shear stress at shear rate 300 s⁻¹, wherein the inoculation temperature is 40° C.; and/or
   (iii) produce more than 1 μg/ml/OD₆₀₀ of EPS under the following conditions:
      Strains are grown in a Chemically Defined Medium supplemented with 2% lactose (w/v) under anaerobic conditions until stationary phase of growth (5-6 h) at 40° C. and constant pH 6.5; cell culture is then centrifuged (13,700×g, 30 min, 4° C.) and the supernatant collected and stored at −20° C. for isolation of EPS/CPS, as described in Example 2.

Any combination of the above-described elements, aspects and embodiments in all possible variations thereof is encompassed by the invention unless otherwise indicated herein or otherwise clearly contradicted by context. Embodiments of the present invention are described below, by way of examples only.

### Deposits and Expert Solutions

The applicant requests that a sample of the deposited microorganisms stated in the table below may only be made available to an expert, until the date on which the patent is granted.

TABLE 1
Deposits made at a Depositary institution having acquired the
status of international depositary authority under the Budapest
Treaty on the International Recognition of the Deposit of Microorganisms
for the Purposes of Patent Procedure: Leibniz Institute DSMZ-
German Collection of Microorganisms and Cell Cultures Inhoffenstr.
7B, 38124 Braunschweig, Germany.

| | ACCESSION | DEPOSIT |
|---|---|---|
| STRAIN | NO. | DATE |
| Streptococcus | DSM 34289 | 2022 Jun. 14 |
| thermophilus | | |
| Streptococcus | DSM 34290 | 2022 Jun. 14 |
| thermophilus | | |
| Streptococcus | DSM 34291 | 2022 Jun. 14 |
| thermophilus | | |

### EXAMPLES

### Example 1: Evaluation of Texture of Fermented Milk Using S. thermophilus Strains Producing the Desired Exocellular Polysaccharide

Strains that may be referred to as “texturing” strains, measured as follows:

Strains incubated overnight in M17 broth containing 2% lactose (or 2% galactose in the case of DSM 34289) were used for milk acidification (1% inoculum) in 200 ml severely heat-treated skim milk added either 2% casein hydrolysate (4 g in 200 ml; Sigma-Aldrich, 22090-500 G) or 0.2% yeast extract (NuCel® 545 MG, batch number 0005115910, batch AD 18 A05030, Procelys). The severely heat-treated skim milk was prepared by reconstituting skim milk powder containing 38% protein, 53% lactose, <1.25 fat, and 3.9% moisture (Arla Foods amba, Denmark) to a level of dry matter of 9.5% and pasteurized at 99° C. for 30 min, followed by cooling to 40° C. The high temperature treatment ensures that the fermentation of this type of milk is not affected by the indigenous enzymes or bacteria. The inoculated milk samples were incubated at 40° C. until pH~4.55 and cooled down to 4° C. The samples were subsequently stirred gently and used to measure the shear stress at different shear rates. Heat-treated skim milk supplemented with 0.2% yeast extract (Media A) or with 2% casein hydrolysate (Media B), as described above, with shear stress at shear rate 300 s⁻¹ of above 17 Pa or above 13.3 Pa, respectively, as a result of fermentation was considered having a good texture.

The rheological properties of the sample were assessed on a rheometer (Anton Paar Physica Rheometer with ASC, Automatic Sample Changer, Anton Paar® GmbH, Austria) by using a bob-cup. The rheometer was set to a constant temperature of 13° C. during the time of measurement. Settings were as follows:
Holding time (to rebuild to somewhat original structure);
5 minutes without any physical stress (oscillation or rotation) applied to the sample;
Oscillation step (to measure the elastic and viscous modulus, G′ and G″, respectively, therefore calculating the complex modulus G*)
   Constant strain=0.3%, frequency (f)=[0.5 . . . 8] Hz
   6 measuring points over 60 s (one every 10 s);
Rotation step (to measure shear stress at 300 1/s);
Two steps were designed:
Shear rate=[0.3-300] 1/s and 2) Shear rate=[275-0.3] 1/s.

Each step contained 21 measuring points over 210 s (on every 10 s). The shear stress at 300 1/s (300 s⁻¹) was chosen for further analysis, as this correlates to mouth thickness when swallowing a fermented milk product.

The S. thermophilus strains of the invention all comprising an active eps gene cluster capable of producing the exocellular polysaccharide structures of the invention clearly show a higher texture measured as an enhanced shear stress as compared to a non-texturing S. thermophilus control strain.

TABLE 2
Shear stress at shear rate 300 s⁻¹ of milk fermented
with strains producing the EPS of the invention.

| STRAIN | MEDIA A | MEDIA B |
|---|---|---|
| DSM 34289 | 36.2 | 41.2 |
| DSM 34290 | 40.9 | 41.4 |
| DSM 34291 | 66.9 | 64.9 |
| S. thermophilus | 17.0 | 13.3 |
| control strain | | |

### Example 2: Evaluation of EPS Production in the Strains of the Invention

Strains were grown in a Chemically Defined Medium supplemented with 2% lactose (w/v) (CDMLac) under anaerobic conditions until stationary phase of growth (5-6 h) at 40° C. and constant pH 6.5. The culture was centrifuged (13700×g, 30 min, 4° C.) and the supernatant collected and stored at −20° C. for isolation of EPS. Culture supernatants were loaded on a 12 kDa cut-off dialysis tubes (Pur-A-Lyzer Mega 12000) and treated with benzonase (25 kU) overnight at 37° C. during dialysis in 50 mM NaNO₃, 2 mM MgSO₄. Proteinase K was subsequently added and sample dialyzed in fresh 50 mM NaNO₃, 2 mM MgSO₄ at 65° C. for 3 h. Two volumes of ice-cold ethanol were added to the crude EPS and allowed to precipitate overnight at −20° C. The sample was centrifuged (3256×g, 30 min at 4° C.) and the ethanol discarded. The pellet was dried at 37° C. during 1 h to evaporate any remaining ethanol, suspended in MilliQ water and pellets dissolved under rotation for 30 min at 4° C. Sample was transferred to a 1.5 ml tube and centrifuged at 20000×g, 15 min at room temperature. The purified EPS was transferred to a clean tube and kept at −20° C. until quantification. Polysaccharide fractions were quantified by the phenol:sulfuric acid method (e.g., as described in DuBois et al. 1956. Colorimetric method for determination of sugars and related substances. Anal Chem 28:350-356) to determine total sugar content. Values in Table 3 are the average of at least 5 technical replicates±standard deviation.

When compared to the Streptococcus thermophilus control strain, the strains of the invention predominantly produce the excreted EPS of the present invention. It was previously thought that higher amounts of EPS would necessarily lead to concomitant increase in viscosity, i.e. higher shear stress. EPS with a structure as claimed are able increase viscosity, i.e., higher shear stress (Example 1).

TABLE 3
Levels of EPS produced by the strains of the invention.

| | EPS level | |
|---|---|---|
| | normalized | |
| | by biomass | EPS levels |
| STRAIN | (μg/mL/OD₆₀₀) | (μg/mL) |
| DSM 34289 | 7 ± 0.4 | 53 ± 3.3 |
| DSM 34290 | 5 ± 0.4 | 25 ± 1.9 |
| DSM 34291 | 12 ± 0.1 | 93 ± 1.0 |
| S. thermophilus | 1 ± 0.2 | 4 ± 1.1 |
| control strain | | |

### Example 3: Determination of Exocellular Polysaccharide Structure

Strains were grown in a chemically defined medium (Otto medium) under anaerobic conditions for 24 h at 40° C. The culture was then centrifuged (10000 g, 1 h, 4° C.) and the supernatant was treated with trichloroacetic acid 80% (w/v) to a final concentration of 20%. The precipitation was allowed to proceed at 4° C. with agitation for 2 h. The mixture was centrifuged (10000×g, 1 h, 4° C.) and the crude EPS was precipitated by the addition to the supernatant of an equal volume of acetone (4° C.). After allowing for overnight precipitation at 4° C. with mild agitation, the sample was centrifuged (10,000 g, 1 h, 4° C.). The pellet was washed three times with 50% acetone (4° C.) and the resulting pellet was suspended in 50 ml distilled water with vigorous agitation. The suspension was subjected to sonication for 5 min leading to a clear solution. Finally, this solution was dialyzed for 12 h against distilled water (5 L) at 4° C. The dialysis was repeated a total of three times, each against fresh distilled water. The content of the dialysis bag was freeze dried and the EPS residue was solubilized in 1 mL D₂O by vigorous agitation and sonication.

For the structural elucidation by NMR, the spectra were acquired on an AVANCE III 800 spectrometer (Bruker, Rheinstetten, Germany) working at a proton operating frequency of 800.33 MHz equipped with a three-channel inverse detection probe (TXI) with pulse-field gradients. Two-dimensional spectra for structural elucidation were acquired at 60° C. using standard Bruker pulse programs. Proton-homonuclear shift correlation spectroscopy (COSY), total correlation spectroscopy (TOCSY), ¹H-¹³C multiplicity edited heteronuclear single quantum coherence spectra (ed-HSQC), and two-dimensional HSQC-TOCSY were used for the complete assignment of all the resonances in the HSQC maps. ³J_{H1,H2} as measured directly from the 1D ¹H spectra and ¹J_{C,H1} as measured from non-decoupled HSQC spectra were used to establish the configurations of the sugar rings. Heteronuclear multiple bond connectivity spectra (HMBC) were acquired to determine the position of glycosidic bonds. The identity of the sugar monomers was established by ¹H and ¹³C chemical shift patterns and, when possible, by the determination of distinct ³J_{H,H·} couplings.

TABLE 4
Exocellular polysaccharides and strains producing them.
DSM 34289
Structure (01)

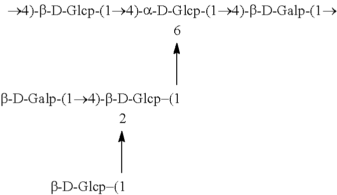

DSM 34290
Structure (02)

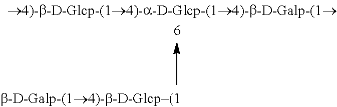

DSM 34291
Structure (03)

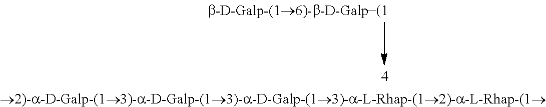

Streptococcus thermophilus control strain
Structure (04)

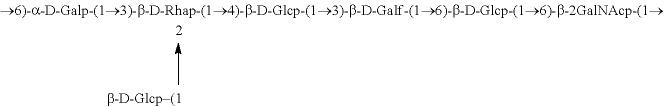

1. A polysaccharide comprising a repeating unit which comprises a backbone as defined by one of the following structures: 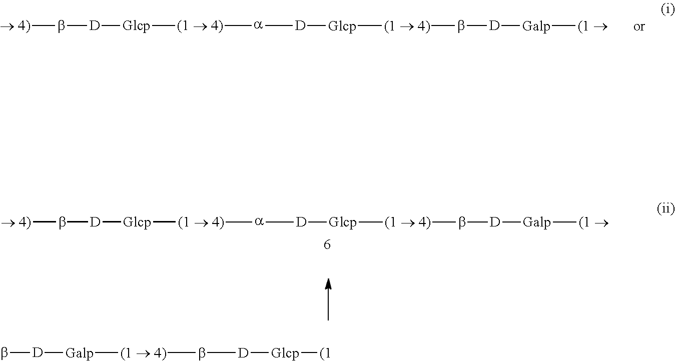
wherein the repeating unit comprising backbone (i) further comprises at least one side chain attached to any of the sugars in the backbone, wherein the side chain comprises or consists of structure (a): 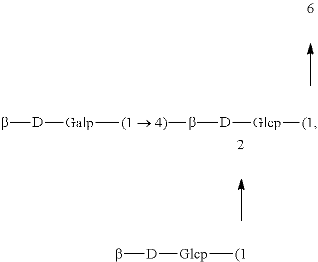
where (1->6) represents the linker to the monosaccharide in the backbone; and
wherein the repeating unit comprising backbone (ii) further comprises at least one side chain attached to any of the sugars in the backbone, wherein the side comprises or consists of structure (b): 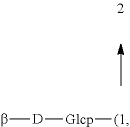
where (1->2) represents the linker to the monosaccharide in the backbone.

2. The polysaccharide according to claim 1, wherein the polysaccharide comprises a repeating unit which comprises or consists of the following structure:
(01)

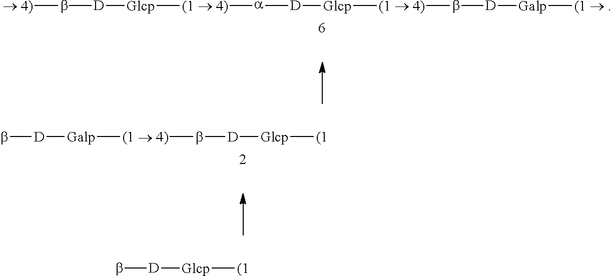

3. The polysaccharide of claim 1, wherein the molecular weight of the polysaccharide is at least 1000 kDa.

4. A lactic acid bacterium strain producing the polysaccharides as defined in claim 1 or a polysaccharide comprising a repeating unit which comprises or consists of any one of the following structures:

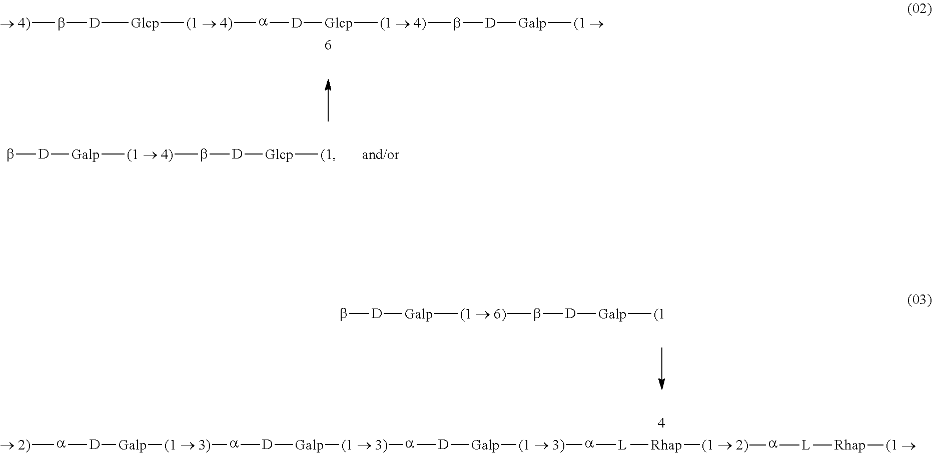

5. The lactic acid bacterium strain of claim 4, wherein the lactic acid bacterium strain is a strain which generates fermented milks having a shear stress of more than 17 Pa measured at shear rate 300 s⁻¹ in the presence of 0.2% yeast extract, measured under following conditions:
200 mL skim milk (38% protein, 53% lactose, <1.25 fat) is heated to 99° C. for 30 min, followed by cooling to inoculation temperature, and inoculated with 2 mL of an overnight culture of the lactic acid bacterium strain in the presence of 0.2% yeast extract, and left at inoculation temperature until pH 4.55 and cooling down to 4° C., followed by gently stirring and measuring the shear stress at shear rate 300 s⁻¹, wherein the inoculation temperature is 40° C.

6. The lactic acid bacterium strain of claim 4, wherein the lactic acid bacterium strain is a strain which generates fermented milks having a shear stress of more than 13.3 Pa measured at shear rate 300 s⁻¹ in the presence of 2% casein hydrolysate, measured under following conditions:
200 mL skim milk (38% protein, 53% lactose, <1.25 fat) is heated to 99° C. for 30 min, followed by cooling to inoculation temperature, and inoculated with 2 mL of an overnight culture of the lactic acid bacterium strain in the presence of 2% casein hydrolysate, and left at inoculation temperature until pH 4.55 and cooling down to 4° C., followed by gently stirring and measuring the shear stress at shear rate 300 s⁻¹, wherein the inoculation temperature is 40° C.

7. The lactic acid bacterium strain of claim 4, wherein the lactic acid bacterium strain is a strain which produces more than 1 μg/mL/OD₆₀₀ of EPS under the following conditions:
Strains are grown in a Chemically Defined Medium supplemented with 2% lactose (w/v) under anaerobic conditions until stationary phase of growth (5-6 h) at 40° C. and constant pH 6.5; cell culture is then centrifuged (13,700×g, 30 min, 4° C.) and the supernatant collected and stored at −20° C. for isolation of EPS, as described in Example 2.

8. The lactic acid bacterium of claim 4, wherein the bacterium is selected from the group of strains consisting of: DSM 34289; DSM 34290; DSM 34291; and mutants and variants thereof with retained or further improved texturing properties.

9. A composition comprising at least one of the polysaccharides as defined in claim 1 or a polysaccharide comprising a repeating unit which comprises or consist of one of the structures (02) or (03).

10. A starter culture comprising at least one of the lactic acid bacterium strain as defined in claim 4.

11. (canceled)

12. A method of producing a product comprising a step in which at least one of the polysaccharides as defined in claim 1 or a polysaccharide comprising a repeating unit which comprises or consist of one of the structures (02) or (03).

13. The method of claim 12 wherein the product is a food or feed product.

14. The method of claim 12, further comprising a step of fermenting a substrate with the lactic acid bacterium strain as defined in any one of claims 4-8, a composition as defined in claim 9, and/or a starter culture as defined in claim 10.

15. The method of claim 12, wherein the method includes use of further lactic acid bacteria; casein hydrolysate; and/or yeast extract.

16. The method of claim 12, wherein the product is a dairy product or a plant-based product.

17. A product comprising at least one of the polysaccharides as defined in claim 1 or a polysaccharide comprising a repeating unit which comprises or consist of one of the structures (02) or (03).

18. The product of claim 17, wherein the product comprises or consists of plant-based and/or mammalian milk.

19. A method for manufacturing a lactic acid bacterium strain wherein the method comprises the following steps:
(a) Providing a lactic acid bacterium strain capable of producing a polysaccharide as defined in claim 1; and
(b) Screening for a lactic acid bacterium strain which is able to:
(i) generate fermented milks having a shear stress of more than 17 Pa measured at shear rate 300 s⁻¹ in the presence of 0.2% yeast extract, measured under following conditions:
200 mL skim milk (38% protein, 53% lactose, <1.25 fat) is heated to 99° C. for 30 min, followed by cooling to inoculation temperature, and inoculated with 2 mL of an overnight culture of the lactic acid bacterium strain in the presence of 0.2% yeast extract, and left at inoculation temperature until pH 4.55 and cooling down to 4° C., followed by gently stirring and measuring the shear stress at shear rate 300 s⁻¹, wherein the inoculation temperature is 40° C.; and/or
(ii) generate fermented milks having a shear stress of more than 13.3 Pa measured at shear rate 300 s⁻¹ in the presence of 2% casein hydrolysate, measured under following conditions:
200 mL skim milk (38% protein, 53% lactose, <1.25 fat) is heated to 99° C. for 30 min, followed by cooling to inoculation temperature, and inoculated with 2 mL of an overnight culture of the lactic acid bacterium strain in the presence of 2% casein hydrolysate, and left at inoculation temperature until pH 4.55 and cooling down to 4° C., followed by gently stirring and measuring the shear stress at shear rate 300 s⁻¹, wherein the inoculation temperature is 40° C.; and/or
(iii) produce more than 1 μg/ml/OD₆₀₀ of EPS under the following conditions:
Strains are grown in a Chemically Defined Medium supplemented with 2% lactose (w/v) under anaerobic conditions until stationary phase of growth (5-6 h) at 40° C. and constant pH 6.5; cell culture is then centrifuged (13,700×g, 30 min, 4° C.) and the supernatant collected and stored at −20° C. for isolation of EPS, as described in Example 2.
